(19) United States
(12) Patent Application Publication
(10) Pub. No.: US 2008/0221503 A1
(43) Pub. Date: Sep. 11, 2008

(54) Method of treating diseases

(76) Inventors: Mark Chak, Brooklyn, NY (US); Alexander Chak, Brooklyn, NY (US)

Correspondence Address: Ilya Zborovsky, 6 Schoolhouse Way, Dix Hills, NY, 11746, US

(21) Appl. No.: 11/715,121

(22) Filed: Mar. 8, 2007

(51) Int. Cl. A61M 5/00

(52) U.S. Cl. 604/19

(57) ABSTRACT

A method of treating diseases includes of acting on an organ which has an unhealthy inclusion, and using for the acting on the organ which has an unhealthy inclusion a pressure which deviates from a normal pressure for the inclusion.

### BACKGROUND OF THE INVENTION

The present invention relates to a method of treating diseases.

Methods of treatment of diseases including action on an unhealthy inclusion in an organ are known in great varieties. It is believed that it would be advisable to further improve the existing methods and to increase the efficiency of treatment.

### SUMMARY OF THE INVENTION

Accordingly, it is an object of the present invention to provide a method of treating diseases, which is a further improvement of the existing solutions.

In keeping with these objects and with others which will become apparent hereinafter, one feature of the present invention resides, briefly stated, a method of treating diseases, comprising the steps of acting on an organ which has an unhealthy inclusion; and using for the acting on the organ which has an unhealthy inclusion a pressure which deviates from a normal pressure for said inclusion.

Another feature of the present invention resides, briefly stated, in applying a pressure to said inclusion in said organ.

A further feature of the present invention resides, briefly stated, wherein said using includes applying a cold to said inclusion in said organ to reduce a pressure on said inclusion in said organ below a normal pressure.

Still a further feature resides, briefly stated, wherein said using includes acting on said inclusion in said organ with heat which increases a pressure on said inclusion and said organ above a normal pressure.

The novel features which are considered as characteristic for the present invention are set forth in particular in the appended claims, The invention itself, however, both as to its construction and its method of operation, together with additional objects and advantages thereof, will be best understood from the following description of specific embodiments when read in connection with the accompanying drawings.

### BRIEF DESCRIPTION OF THE DRAWINGS

FIG. 1 is a view illustrating a method of treating diseases in accordance with one embodiment of the present invention;

FIG. 2 is a view illustrating a method of treating diseases in accordance with another embodiment of the present invention;

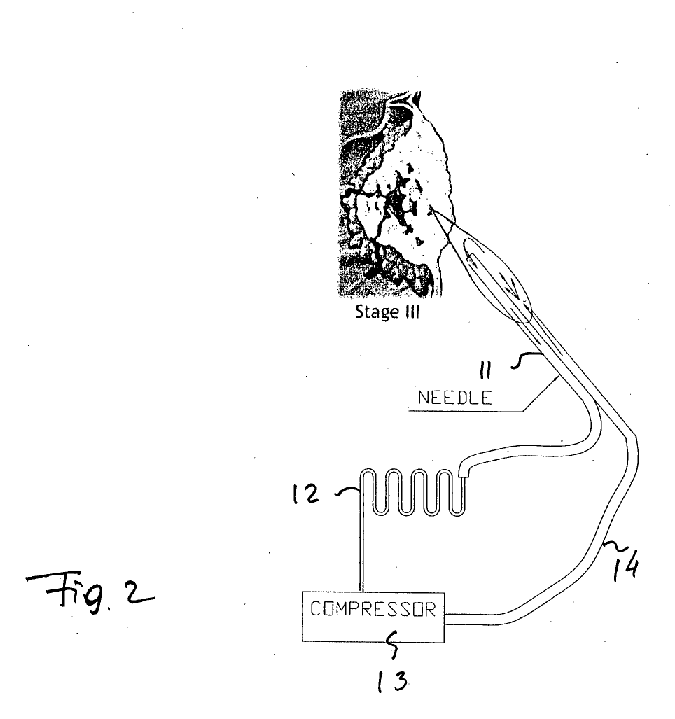

FIG. 3 is a view illustrating a method of treating diseases in accordance with a further embodiment of the present invention.

### DESCRIPTION OF THE PREFERRED EMBODIMENTS

In accordance with the present invention, in an organ having an unhealthy inclusion a pressure is generated which deviates from a normal pressure characteristic for said organ and said inclusion.

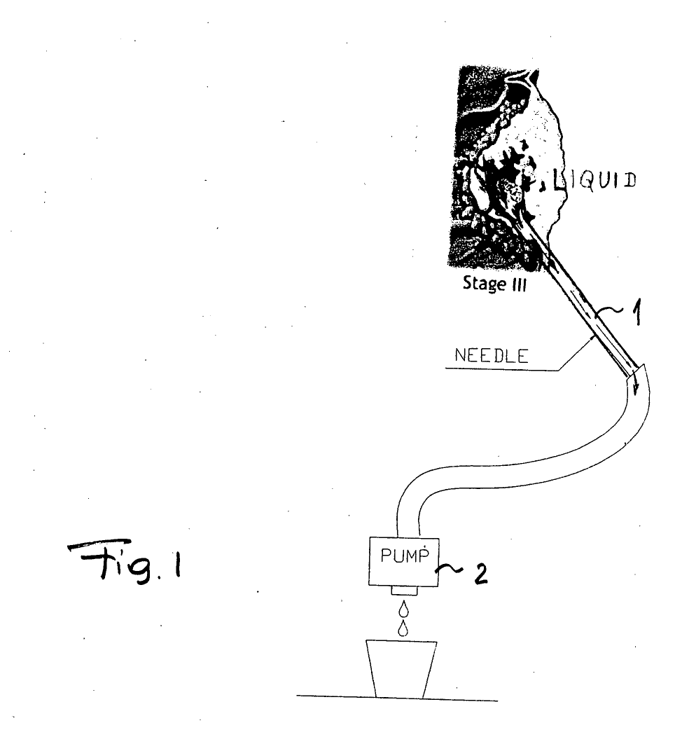

As shown in FIG. 1, in an organ having for example cancerous cells, a needle 1 is introduced which is connected with a pump 2. By means of the pump 2 through the needle 1 a liquid is pumped out from the area of the unhealthy inclusion or in particular the cancerous cells. As a result, a pressure is reduced below a normal pressure. The reduced pressure which is abnormal for the unhealthy inclusion suppresses the unhealthy inclusion and causes recovery of the organ.

In accordance with another embodiment of the present invention, a needle 11 is introduced into an organ with an unhealthy inclusion, through which a liquid is withdrawn from the organ. A cooling medium from a cooling aggregate 12 is supplied by a compressor 13 through a pipe 14 into the area of the unhealthy inclusion. The unhealthy inclusion is subjected to a high level cold to freeze it. As a result, a pressure in the unhealthy inclusion significantly lowers and the unhealthy inclusion is suppressed, which leads to recovery of the organ.

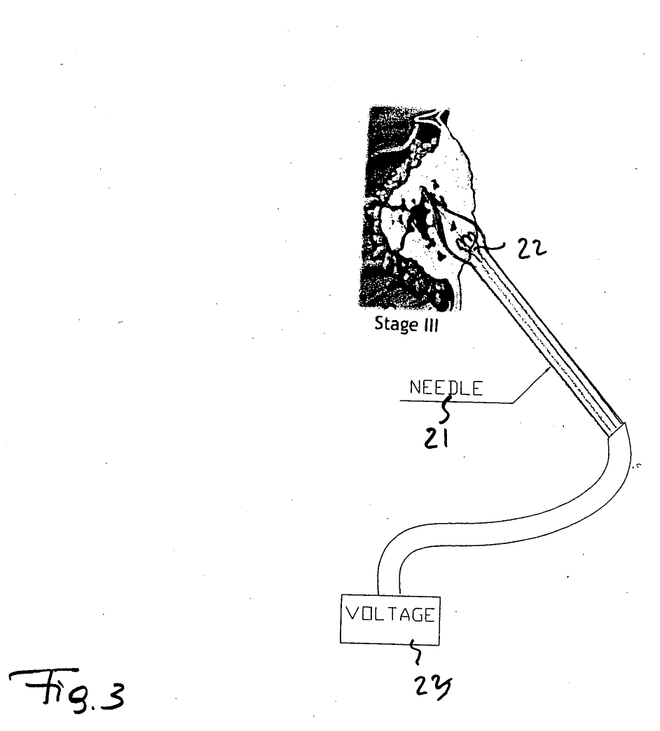

In accordance with a further embodiment shown in FIG. 3, a needle 21 is introduced into an organ with an unhealthy inclusion. A heating device 22 supplied with power from a source 23 heats the area of the unhealthy inclusion in an organ. The pressure in the unhealthy inclusion increases above normal and the unhealthy inclusion is suppressed, thus leading to a recovery of the organ.

The inventive method can be used for human beings and also for animals.

It will be understood that each of the elements described above, or two or more together, may also find a useful application in other types of methods differing from the types described above.

While the invention has been illustrated and described as embodied in a method of treating diseases, it is not intended to be limited to the details shown, since various modifications and structural changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed as new and desired to be protected by Letters Patent is set forth in the appended claims:

1. A method of treating diseases, comprising the steps of acting on an organ which has an unhealthy inclusion; and using for the acting on the organ which has an unhealthy inclusion a pressure which deviates from a normal pressure for said inclusion.

2. A method as defined in claim 1, wherein said using includes applying a pressure to said inclusion in said organ.

3. A method as defined in claim 1, wherein said using includes applying a cold to said inclusion in said organ to reduce a pressure on said inclusion in said organ below a normal pressure.

4. A method as defined in claim 1, wherein said using includes acting on said inclusion in said organ with heat which increases a pressure on said inclusion in said organ above a normal pressure.
